(19) United States
(12) Patent Application Publication
(10) Pub. No.: US 2022/0408780 A1
(43) Pub. Date: Dec. 29, 2022

(54) A METHOD EFFECTIVE FOR TREATING ULCERATIVE COLITIS

(71) Applicant: THE E. WOLFSON MEDICAL CENTER, Holon (IL)

(72) Inventors: Arie LEVINE, Mazkeret Batya (IL); Chen SARBAGILI SHABAT, Rehovot (IL); Rotem SIGALL BONEH, Holon (IL)

(21) Appl. No.: 17/798,665

(22) PCT Filed: Feb. 10, 2021
(86) PCT No.: PCT/IL2021/050163

§ 371 Date: Aug. 10, 2022

Related U.S. Application Data
(60) Provisional application No. 62/972,663, filed on Feb. 11, 2020.

(51) Int. Cl. A23L 33/00; G16H 20/60

(52) U.S. Cl. CPC A23L 33/30; G16H 20/60

(57) ABSTRACT

A method for providing a two stage diet for an Ulcerative Colitis patient, and therapeutic meals for same, the method comprising stage 1 which is provided in weeks 0-6, and stage 2 which is provided in weeks 7-12; the diet described herein providing dietary components with reduced content of animal fat, dairy products, gluten, taurine, yeast, sulfites, and emsulsifiers.

### FIELD OF THE INVENTION

The invention relates to a method for induction of remission of Ulcerative Colitis (UC). More specifically, the method discloses a diet that reduces exposure to dietary components shown to induce inflammation in Ulcerative Colitis patients.

### BACKGROUND OF THE INVENTION

Ulcerative colitis (UC) is a chronic inflammatory disease primarily involving the colon. It has long been considered to be due to a dysregulated immune response targeting the colon, and involves unknown environmental factors (1). Recent studies have highlighted several characteristics which may suggest that UC is associated with alterations of the microbiota (2-5), decreased short chain fatty acid producing bacteria (13) and defective production of short chain fatty acids (6-7) and an impaired mucous layer or goblet cells (8). However at present, no effective therapy targets the microbiota or its interaction with the colonic epithelium. Moreover, it has been shown that certain dietary patterns in humans and rodent models may induce dysbiosis, and that colitis may be associated with bacteria from the Proteobacteria or sulphide reducing bacteria (SRB) or absence of short chain fatty acid producing bacteria such as Fecalibacterium prausnitzii and Roseburia. (5-7,13). An illustration of the relationship between host gut and microbiome is shown in FIG. 1. Further support for targeting the microbiota includes several studies demonstrating that antibiotics might be helpful for severe refractory colitis (13-17). Development of treatment strategies that target the microbiota could reduce exposure to immune suppression, and add new therapeutic strategies that do not exist at present.

Although diet has a significant impact on the composition of the microbiota (5,18) no dietary intervention to date has proven effective for induction of remission (19-21). We hypothesized that ulcerative colitis is caused by a series of events involving alterations in goblet cells or mucin production, dysbiosis, with sulfate or sulfide reducing bacteria or other pathobionts combined with defective production of short chain fatty acids. We also hypothesized that interventions that target colonic bacteria such as a diet that we developed that reduces exposure to dietary ingredients that allow sulfide reducing bacteria to thrive, or that impair the mucous layer, coupled with dietary products that enhance butyrate producing bacteria or butyrate production, could change the metabolic function and dysbiosis present in UC.

Thus, it remains necessary to provide a long-term solution for treating Ulcerative Colitis.

### SUMMARY

The current invention provides a method for providing a two stage diet for an Ulcerative Colitis patient, the method comprising stage 1 which is provided in weeks 0-6, and stage 2 which is provided in weeks 7-12; wherein stage 1 comprises the step of consuming the following food products; at least one portion of fresh chicken breast three times a week, two peeled potatoes a day, 2 bananas a day, 1 apple a day, at least one portion of fresh salad a day from spinach or lettuce leaves with daily allowed fruit or vegetables, one of the following: one egg a day for patients below 40 kg, or two eggs a day for vegetarian patients and patients above 40 kg; unlimited amounts of products selected from the group consisting of: white rice, rice noodles/rice papers, rice flour, olive oil, canola oil, salt, pepper, paprika, cinnamon, curcumin, turmeric, fresh herbs, fresh ginger, fresh lemon juice, fresh tomato sauce, baking soda, water, soda, herbal teas, chamomile tea; no more by quantity than the following: tofu, 200 grams once a week from week 4, 3 additional eggs a week for cooking or baking, plain unflavored regular (3-4%) fat yoghurt 3 times a week, oatmeal up to ¼ cup (dry) a day, almond flour, up to ¼ cup weekly, cooked durum pasta, up to 200 grams, twice a week, cooked quinoa, up to 4 tablespoons a day, legumes, selected from the group consisting of: lentils, peas or cooked green beans, up to 4 tablespoons a day, fresh melon selected from the group consisting of cantaloupe, honeydew or watermelon, up to one slice daily, fresh berries daily selected from the group consisting of five strawberries, five raspberries or ten blueberries, one fresh fruit per day, selected from the group consisting of: 1 kiwi, 10 seedless grapes or 1 pear, 2 tomatoes daily, 2 cucumbers daily, 3 lettuce leaves daily, 1 cup spinach leaves daily, 1 carrot daily, 1 zucchini daily, 3 broccoli or cauliflower florets or clusters daily, 1 avocado daily, 100 grams pumpkin daily, ½ red pepper daily, 6 unprocessed walnut halves or unprocessed unroasted almonds a day, one glass of freshly squeezed orange juice per day, pure honey for cooking or desserts, up to 3 tablespoons a day and table sugar for cooking or tea, up to 3 teaspoons a day;

further wherein stage 2 comprises the step of consuming the following food products: one portion of fresh chicken breast 3 times a week, one of the following: one egg a day for patients below 40 kg, or two eggs a day for vegetarian patients and patients above 40 kg, 2 fresh peeled potatoes or 1 peeled potato and 1 peeled sweet potato, daily, 1 banana, 1 apple (not cooked), and one additional fruit daily, selected from the group consisting of; 1 slice melon (cantaloupe honeydew), fresh berries daily, selected from the group consisting of: 5 strawberries, 5 raspberries or 10 blueberries and one portion of salad daily, comprising a combination of allowed vegetables, fruits and nuts; unlimited amounts of products selected from the group consisting of: white rice, rice noodles/rice papers, rice flour, lettuce/roquette, arugula, olive oil, canola oil, water, soda, herbal teas, chamomile tea, any home-made juice smoothie, salt, pepper, paprika, cinnamon stick, curcumin, turmeric, fresh herbs, fresh ginger, fresh lemon juice, fresh tomato sauce, unsweetened cocoa powder without additives and sulfite-free dark raisins for baking; no more by quantity than the following: Tofu 200 grams, once a week, 1 plain unflavored regular (3-4%) fat yoghurt daily, 1 additional egg a day, oatmeal, up to ½ a cup per day, wheat or spelt flour for baking, up to ½ cup once a week, 1 additional sweet potato daily, cooked quinoa, up to 4 tablespoons a day, legumes, selected from the group consisting of: lentils, peas, green beans, or dried chickpeas, up to 5 tablespoons a day, 1 slice of whole grain bread or ½ a cup of bread crumbs daily, cooked durum pasta, 200 grams, up to 2 times a week, one corn cob a day, fresh melon selected from the group consisting of cantaloupe, honeydew or watermelon, up to one slice daily, fresh berries selected from the group consisting of five strawberries, five raspberries or ten blueberries, a day, one fresh fruit a day, selected from the group consisting of: 1 kiwi, 10 seedless grapes or 1 pear, 4 cubes of mango, 1 slice pineapple, 1 tangerine or one orange, 2 tomatoes daily, 2 cucumbers daily, 1 carrot daily, 1 zucchini daily, 3 broccoli or cauliflower florets or clusters daily, 1 avocado daily, 100 grams pumpkin, ½ red, yellow or green bell pepper a day, 4 fresh button mushrooms a day, 6 walnut halves or 6 almonds a day, one glass of freshly squeezed orange juice per day, pure honey for cooking, up to 3 tablespoons a day and sugar for tea or cooking, up to 3 teaspoons a day;

It is another object of the current invention to disclose the method as defined in any of the above, wherein the patient is suffering from mild to moderate Ulcerative Colitis.

It is another object of the current invention to disclose the method as defined in any of the above wherein the patient is selected from the group consisting of children, young adults, and any combination thereof.

It is another object of the current invention to disclose the method as defined in any of the above wherein the fresh herbs are selected from the group consisting of mint leaves, oregano, coriander, rosemary, sage, basil, thyme and any combination thereof.

It is another object of the current invention to disclose the method as defined in any of the above, wherein the almonds or walnuts are unsalted and unprocessed.

It is another object of the current invention to disclose the method as defined in any of the above, wherein the food products of stage 1 and stage 2 are fresh.

It is another object of the current invention to disclose the method as defined in any of the above, wherein the avocado is to be consumed over at least two meals.

It is another object of the current invention to disclose the method as defined in any of the above, wherein the two peeled potatoes are cooked and refrigerated before consumption.

It is another object of the current invention to disclose the method as defined in any of the above, wherein the herbal teas may further comprise a slice of fresh lemon, a slice of fresh lime, a slice of fresh orange or mint leaves.

It is another object of the current invention to disclose the method as defined in any of the above, wherein the food products may be processed in a manner selected from the group consisting of grilling, frying, baking, boiling, broiling, and any combination thereof.

It is another object of the current invention to disclose the method as defined in any of the above, wherein the food products may be deep fried, no more than once a week during stage 1, and no more than twice a week during stage 2.

It is another object of the current invention to disclose the method as defined in any of the above, wherein a supplemental dose of vitamin D of at least 1,600 IU/day is added to the food products.

It is another object of the current invention to disclose the method as defined in any of the above, wherein stage 1 and stage 2 additionally comprise the step of not consuming the following foods: red meat, pork, turkey, processed meat, seafood, fish, smoked meats and fish, all dairy products except unflavored yoghurt 3-4% fat, non-dairy milk products, breads and baked goods of any kind, gluten free products not listed above, potato or corn flour, and any flour not listed as allowed, frozen potatoes or yams, corn during stage 1, legumes, comprising beans, chickpeas and soybeans during stage 1 and white beans during stage 2, margarine, any cooking oils except olive and canola oil, non-dairy creamers or cream, coconut oil, butter, dried fruits, canned fruits, all fruits not on the allowed list, all vegetables not on the allowed list, walnuts from mixes or trail mix, all nuts except unprocessed walnuts and almonds, soft drinks, fruit juices and sweetened beverages, alcoholic beverages, coffee, sauces, spreads, salad dressing, syrups and jams of any kind, canned products, packaged snacks, candy, chocolate, cakes, cookies, chewing gum, artificial sweeteners, mixed spices, ice-cream and yeast for baking.

The current invention further provides a range of food products for an Ulcerative Colitis remission diet; the range of food products comprise a first range of food products and a second range of food products; the first range of food products is adapted to be consumed during weeks 0-6 of the diet, and second range of products is adapted to be consumed in weeks 7-12; wherein the first range of food products comprise the following food products: at least one portion of fresh chicken breast three times a week, two peeled potatoes a day, 2 bananas a day, 1 apple a day, at least one portion of fresh salad a day from spinach or lettuce leaves with daily allowed fruit or vegetables, one of the following: one egg a day for patients below 40 kg, or two eggs a day for vegetarian patients and patients above 40 kg; unlimited amounts of products selected from the group consisting of: white rice, rice noodles/rice papers, rice flour, olive oil, canola oil, salt, pepper, paprika, cinnamon, curcumin, turmeric, fresh herbs, fresh ginger, fresh lemon juice, fresh tomato sauce, baking soda, water, soda, herbal teas, chamomile tea; no more by quantity than the following: tofu, 200 grams once a week from week 4, 3 additional eggs for cooking or baking per week, plain unflavored regular (3-4%) fat yoghurt 3 times a week, oatmeal up to ¼ cup (dry) a day, almond flour, up to ¼ cup weekly, cooked durum pasta, up to 200 grams, twice a week, cooked quinoa, up to 4 tablespoons a day, legumes, selected from the group consisting of: lentils, peas or cooked green beans, up to 4 tablespoons a day, fresh melon selected from the group consisting of cantaloupe, honeydew or watermelon, up to one slice daily, fresh berries selected from the group consisting of five strawberries, five raspberries or ten blueberries, a day, one fresh fruit a day, selected from the group consisting of: 1 kiwi, 10 seedless grapes or 1 pear, 2 tomatoes daily, 2 cucumbers daily, 3 lettuce leaves daily, 1 cup spinach leaves daily, 1 carrot daily, 1 zucchini daily, 3 broccoli or cauliflower florets or clusters daily, 1 avocado daily, 100 grams pumpkin daily, ½ red pepper daily, 6 unprocessed walnut halves or unprocessed unroasted almonds a day, one glass of freshly squeezed orange juice per day, pure honey for cooking or desserts, up to 3 tablespoons a day and table sugar for cooking or tea, up to 3 teaspoons a day; further wherein the second range of food products comprise the following food products: one portion of fresh chicken breast 3 times a week, one of the following: one egg a day for patients below 40 kg, or two eggs a day for vegetarian patients and patients above 40 kg, 2 fresh peeled potatoes or 1 potato and 1 sweet potato, daily, 1 banana, 1 apple (not cooked), and one additional fruit daily, selected from the group consisting of: 1 slice melon (cantaloupe honeydew), fresh berries daily, selected from the group consisting of: 5 strawberries, 5 raspberries or 10 blueberries, one portion of salad daily, comprising a combination of allowed vegetables, fruits and nuts; unlimited amounts of products selected from the group consisting of: white rice, rice noodles/rice papers, rice flour, lettuce/roquette, arugula, olive oil, canola oil, water, soda, herbal teas, chamomile tea, any home-made juice smoothie, salt, pepper, paprika, cinnamon stick, curcumin, turmeric, fresh herbs, fresh ginger, fresh lemon juice, fresh tomato sauce, unsweetened cocoa powder without additives, sulfite-free dark raisins for baking; no more by quantity than the following: Tofu 200 grams, once a week, 1 plain unflavored regular (3-4%) fat yoghurt daily, 1 additional egg, oatmeal, up to ½ a cup per day, wheat or spelt flour for baking, up to ½ cup once a week, 1 additional sweet potato daily, cooked quinoa, up to 4 tablespoons a day, legumes, selected from the group consisting of: lentils, peas, green beans, or dried chickpeas, up to 5 tablespoons a day, 1 slice of whole grain bread, or M a cup of bread crumbs daily, cooked durum pasta, 200 grams, up to 2 times a week, one corn cob a day, fresh melon selected from the group consisting of cantaloupe, honeydew or watermelon, up to one slice daily, fresh berries daily, selected from the group consisting of five strawberries, five raspberries or ten blueberries, one fresh fruit a day, selected from the group consisting of: 1 kiwi, 10 seedless grapes or 1 pear, 4 cubes of mango, 1 slice pineapple, 1 tangerine or one orange, 2 tomatoes daily, 2 cucumbers daily, 1 carrot daily, 1 zucchini daily, 3 broccoli or cauliflower florets or clusters daily, 1 avocado daily, 100 grams pumpkin, ½ red, yellow or green bell pepper a day, 4 fresh button mushrooms a day, 6 walnut halves or 6 almonds a day; one glass of freshly squeezed orange juice per day, pure honey for cooking or desserts, up to 3 tablespoons a day and table sugar for cooking or tea, up to 3 teaspoons a day;

It is another object of the current invention to disclose the range of foods as defined in any of the above, wherein the range of food is adapted to be provided to a patient selected from a group consisting of: young adults, children and any combination thereof.

It is another object of the current invention to disclose the range of foods as defined in any of the above, wherein the range of food is adapted to be provided to a patient suffering from mild to moderate Ulcerative Colitis.

It is another object of the current invention to disclose the range of foods as defined in any of the above, wherein the fresh herbs are selected from the group consisting of mint leaves, oregano, rosemary, sage, basil, thyme and any combination thereof.

It is another object of the current invention to disclose the range of foods as defined in any of the above, wherein the almonds or walnuts are unsalted and unprocessed.

It is another object of the current invention to disclose the range of foods as defined in any of the above, wherein the food products of stage 1 and stage 2 are fresh.

It is another object of the current invention to disclose the range of foods as defined in any of the above, wherein the avocado is to be consumed over at least two meals.

It is another object of the current invention to disclose the range of foods as defined in any of the above, wherein the two peeled potatoes are cooked and refrigerated before consumption.

It is another object of the current invention to disclose the range of foods as defined in any of the above, wherein the herbal teas may further comprise a slice of fresh lemon, a slice of fresh lime, a slice of fresh orange or mint leaves.

It is another object of the current invention to disclose the range of foods as defined in any of the above, wherein the food products may be processed in a manner selected from the group consisting of grilling, frying, baking, boiling, broiling, and any combination thereof.

It is another object of the current invention to disclose the range of foods as defined in any of the above, wherein the food products may be deep fried no more than once a week during stage 1, and no more than twice a week during stage 2.

It is another object of the current invention to disclose the range of foods as defined in any of the above, wherein a supplemental dose of vitamin D of at least 1,600 IU/day is added to the food products.

It is another object of the current invention to disclose the range of foods as defined in any of the above, wherein stage 1 and stage 2 additionally comprise the step of not consuming the following foods: red meat, pork, turkey, processed meat, seafood, fish, smoked meats and fish, all dairy products except unflavored yoghurt 3-4% fat, non-diary milk products, breads and baked goods of any kind, gluten free products not listed above, potato or corn flour, and any flour not listed as allowed, frozen potatoes or yams, corn during stage 1, legumes, comprising beans, chickpeas and soybeans during stage 1 and white beans during stage 2, margarine, any cooking oils except olive and canola oil, non-dairy creamers or cream, coconut oil, butter, dried fruits, canned fruits, all fruits not on the allowed list, all vegetables not on the allowed list, walnuts from mixes or trail mix, all nuts except unprocessed walnuts and almonds, soft drinks, fruit juices and sweetened beverages, alcoholic beverages, coffee, sauces, spreads, salad dressing, syrups and jams of any kind, canned products, packaged snacks, candy, chocolate, cakes, cookies, chewing gum, artificial sweeteners, mixed spices, ice-cream, yeast for baking.

The current invention further provides a method of preventing Ulcerative Colitis to be followed for at least 12 weeks, the method comprising the following steps of limiting all food intake to a first range of food products in weeks 0-6 and to a second range of food products in weeks 7-12; wherein the first range of food products comprise the following food products; at least one portion of fresh chicken breast three times a week, two potatoes a day, 2 bananas a day, 1 apple a day, at least one portion of fresh salad a day from spinach or lettuce leaves with daily allowed fruit or vegetables and one of the following: one egg a day for patients below 40 kg, or two eggs a day for vegetarian patients and patients above 40 kg; unlimited amounts of products selected from the group consisting of: white rice, rice noodles/rice papers, rice flour, olive oil, canola oil, salt, pepper, paprika, cinnamon, curcumin, turmeric, fresh herbs, fresh ginger, fresh lemon juice, fresh tomato sauce, baking soda, water, soda, herbal teas, chamomile tea: no more by quantity than the following: tofu, 200 grams once a week from week 4, 3 additional eggs for cooking or baking per week, plain unflavored regular (3-4%) fat yoghurt 3 times a week, oatmeal up to ¼ cup (dry) a day, almond flour, up to ¼ cup weekly, cooked durum pasta, up to 200 grams, twice a week, cooked quinoa, up to 4 tablespoons a day, legumes, selected from the group consisting of: lentils, peas or cooked green beans, up to 4 tablespoons a day, fresh melon selected from the group consisting of cantaloupe, honeydew or watermelon, up to one slice daily, fresh berries selected from the group consisting of five strawberries, five raspberries or ten blueberries a day, one fresh fruit daily, selected from the group consisting of: 1 kiwi, 10 seedless grapes or 1 pear, 2 tomatoes daily, 2 cucumbers daily, 3 lettuce leaves daily, 1 cup spinach leaves daily, 1 carrot daily, 1 zucchini daily, 3 broccoli or cauliflower florets or clusters daily, 1 avocado daily, 100 grams pumpkin daily, ½ red pepper daily, 6 unprocessed walnut halves or unprocessed unroasted almonds a day, one glass of freshly squeezed orange juice per day, pure honey for cooking or desserts, up to 3 tablespoons a day and table sugar for cooking or tea up to 3 teaspoons a day;

further wherein the second range of food products comprise the following food products: one portion of fresh chicken breast 3 times a week, one of the following: one egg a day for patients below 40 kg, or two eggs a day for vegetarian patients and patients above 40 kg, 2 fresh peeled potatoes or 1 potato and 1 sweet potato daily, 1 banana, 1 apple (not cooked), and one additional fruit daily, selected from the group consisting of: 1 slice melon (cantaloupe or honeydew), fresh berries selected from the group consisting of: 5 strawberries, 5 raspberries or 10 blueberries, one portion of salad daily, comprising a combination of allowed vegetables, fruits and nuts; unlimited amounts of products selected from the group consisting of: white rice, rice noodles/rice papers, rice flour, lettuce/roquette, arugula, olive oil, canola oil, water, soda, herbal teas, chamomile tea, any home-made juice smoothie, salt, pepper, paprika, cinnamon stick, curcumin, turmeric, fresh herbs, fresh ginger, fresh lemon juice, fresh tomato sauce, unsweetened cocoa powder without additives, sulfite-free dark raisins for baking; no more by quantity than the following: Tofu 200 grams, once a week, 1 plain unflavored regular (3-4%) fat yoghurt daily, 1 additional egg, oatmeal, up to ½ cup per day, wheat or spelt flour for baking, up to ½ cup once a week, 1 additional sweet potato daily, cooked quinoa, up to 4 tablespoons a day, legumes, selected from the group consisting of: lentils, peas, green beans, or dried chickpeas, up to 5 tablespoons a day, 1 slice of whole grain bread or 2 a cup of bread crumbs daily, cooked durum pasta, 200 grams, up to 2 times a week, one corn cob a day, fresh melon selected from the group consisting of cantaloupe, honeydew or watermelon, up to one slice daily, fresh berries selected from the group consisting of five strawberries, five raspberries or ten blueberries a day, one fresh fruit a day, selected from the group consisting of: 1 kiwi, 10 seedless grapes or 1 pear, 4 cubes of mango, 1 slice pineapple, 1 tangerine or one orange, 2 tomatoes daily, 2 cucumbers daily, 1 carrot daily, 1 zucchini daily, 3 broccoli or cauliflower florets or clusters daily, 1 avocado daily, 100 grams pumpkin, ½ red, yellow or green bell pepper a day, 4 fresh button mushrooms a day, 6 walnut halves or 6 almonds a day, one glass of freshly squeezed orange juice per day, pure honey for cooking or desserts, up to 3 tablespoons a day and table sugar for cooking or tea, up to 3 teaspoons a day;

It is another object of the current invention to disclose the method as defined in any of the above, wherein the limiting of food intake is carried out by a patient suffering from mild to moderate Ulcerative Colitis.

It is another object of the current invention to disclose the method as defined in any of the above, wherein the limiting of food intake is carried out by a patient selected from the group consisting of: young adults, children and any combination thereof.

It is another object of the current invention to disclose the method as defined in any of the above, wherein the fresh herbs are selected from the group consisting of mint leaves, oregano, coriander, rosemary, sage, basil, thyme and any combination thereof.

It is another object of the current invention to disclose the method as defined in any of the above, wherein the almonds or walnuts are unsalted and unprocessed.

It is another object of the current invention to disclose the method as defined in any of the above, wherein the food products of stage 1 and stage 2 are fresh.

It is another object of the current invention to disclose the method as defined in any of the above, wherein the avocado is to be consumed over at least two meals.

It is another object of the current invention to disclose the method as defined in any of the above, wherein the two peeled potatoes are cooked and refrigerated before consumption.

It is another object of the current invention to disclose the method as defined in any of the above, wherein the herbal teas may further comprise a slice of fresh lemon, a slice of fresh lime, a slice of fresh orange or mint leaves.

It is another object of the current invention to disclose the method as defined in any of the above, wherein the food products may be processed in a manner selected from the group consisting of grilling, frying, baking, boiling, broiling, and any combination thereof.

It is another object of the current invention to disclose the method as defined in any of the above, wherein the food products may be deep fried no more than once a week during stage 1, and no more than twice a week during stage 2.

It is another object of the current invention to disclose the method as defined in any of the above, wherein a supplemental dose of vitamin D of at least 1,600 IU/day is added to the food products.

It is another object of the current invention to disclose the method as defined in any of the above, wherein stage 1 and stage 2 additionally comprise the step of not consuming the following foods: red meat, pork, turkey, processed meat, seafood, fish, smoked meats and fish, all dairy products except unflavored yoghurt 3-4% fat, non-diary milk products, breads and baked goods of any kind, gluten free products not listed above, potato or corn flour, and any flour not listed as allowed, frozen potatoes or yams, corn during stage 1, legumes, comprising beans, chickpeas and soybeans during stage 1 and white beans during stage 2, margarine, any cooking oils except olive and canola oil, non-dairy creamers or cream, coconut oil, butter, dried fruits, canned fruits, all fruits not on the allowed list, all vegetables not on the allowed list, walnuts from mixes or trail mix, all nuts except unprocessed walnuts and almonds, soft drinks, fruit juices and sweetened beverages, alcoholic beverages, coffee, sauces, spreads, salad dressing, syrups and jams of any kind, canned products, packaged snacks, candy, chocolate, cakes, cookies, chewing gum, artificial sweeteners, mixed spices, ice-cream, yeast for baking.

The current invention further provides a daily prepackaged therapeutic meal of a daily diet for consumption to be consumed daily for at least 12 weeks by an Ulcerative Colitis patient; the prepackaged therapeutic meal comprising food products for daily consumption; the food products comprising a first range of food products in the prepackaged therapeutic meal adapted to be consumed in weeks 0-6 and a second range of food products in the prepackaged therapeutic meal adapted to be consumed in weeks 7-12; wherein the first range of food products comprise the following food products: at least one portion of fresh chicken breast three times a week, two peeled potatoes a day, 2 bananas a day, 1 apple a day, at least one portion of fresh salad a day from spinach or lettuce leaves with daily allowed fruit or vegetables, one of the following: one egg a day for patients below 40 kg, or two eggs a day for vegetarian patients and patients above 40 kg; unlimited amounts of products selected from the group consisting of: white rice, rice noodles/rice papers, rice flour, olive oil, canola oil, salt, pepper, paprika, cinnamon, curcumin, turmeric, fresh herbs, fresh ginger, fresh lemon juice, fresh tomato sauce, baking soda, water, soda, herbal teas, chamomile tea; no more by quantity than the following: tofu, 200 grams once a week from week 4, 3 additional eggs for cooking or baking, plain unflavored (3-4%) fat yoghurt 3 times a week, oatmeal up to ¼ cup (dry) a day, almond flour, up to ¼ cup weekly, cooked durum pasta, up to 200 grams, twice a week, cooked quinoa, up to 4 tablespoons a day, legumes, selected from the group consisting of: lentils, peas or cooked green beans, up to 4 tablespoons a day, fresh melon selected from the group consisting of cantaloupe, honeydew or watermelon, up to one slice daily, fresh berries selected from the group consisting of five strawberries, five raspberries or ten blueberries a day, one fresh fruit selected from the group consisting of: 1 kiwi, 10 seedless grapes or 1 pear, 2 tomatoes daily, 2 cucumbers daily, 3 lettuce leaves daily, 1 cup spinach leaves daily, 1 carrot daily, 1 zucchini daily, 3 broccoli or cauliflower florets or clusters daily, 1 avocado daily, 100 grams pumpkin daily, 2 red pepper daily, 6 unprocessed walnut halves or unprocessed unroasted almonds a day, one glass of freshly squeezed orange juice per day, pure honey for cooking or desserts, up to 3 tablespoons a day and table sugar for cooking or tea, up to 3 teaspoons a day;

further wherein the second range of food products comprise the following food products: one portion of fresh chicken breast 3 times a week, one of the following: one egg a day for patients below 40 kg, or two eggs a day for vegetarian patients and patients above 40 kg, 2 fresh potatoes or 1 potato and 1 sweet potato daily, 1 banana, 1 apple (not cooked), and one additional fruit daily, selected from the group consisting of: 1 slice melon (cantaloupe or honeydew), fresh berries (5 strawberries, 5 raspberries or 10 blueberries), one portion of salad daily, comprising a combination of allowed vegetables, fruits and nuts; unlimited amounts of products selected from the group consisting of: white rice, rice noodles/rice papers, rice flour, lettuce/roquette, arugula, olive oil, canola oil, water, soda, herbal teas, chamomile tea, any home-made juice smoothie, salt, pepper, paprika, cinnamon stick, curcumin, turmeric, fresh herbs, fresh ginger, fresh lemon juice, fresh tomato sauce, unsweetened cocoa powder without additives, sulfite-free dark raisins for baking; no more by quantity than the following: Tofu 200 grams, once a week, 1 plain unflavored regular (3-4%) fat yoghurt daily, 1 additional egg daily, oatmeal, up to ½ a cup per day, wheat or spelt flour for baking, up to ½ cup once a week, 1 additional sweet potato daily, cooked quinoa, up to 4 tablespoons a day, legumes, selected from the group consisting of: lentils, peas, green beans, or dried chickpeas, up to 5 tablespoons a day, 1 slice of whole grain bread, or ½ a cup of bread crumbs daily, cooked durum pasta, 200 grams, up to 2 times a week, one corn cob a day, fresh melon selected from the group consisting of cantaloupe, honeydew or watermelon, up to one slice daily, fresh berries selected from the group consisting of five strawberries, five raspberries or ten blueberries a day, one fresh fruit a day, selected from the group consisting of: 1 kiwi, 10 seedless grapes or 1 pear, 4 cubes of mango, 1 slice pineapple, 1 tangerine or one orange, 2 tomatoes daily, 2 cucumbers daily, 1 carrot daily, 1 zucchini daily, 3 broccoli or cauliflower florets or clusters daily, 1 avocado daily, 100 grams pumpkin, 2 red, yellow or green bell pepper a day, 4 fresh button mushrooms a day, 6 walnut halves or 6 almonds a day; one glass of freshly squeezed orange juice per day, pure honey for cooking or desserts, up to 3 tablespoons a day and table sugar for cooking or tea, up to 3 teaspoons a day;

It is another object of the current invention to disclose the daily prepackaged therapeutic meal as defined in any of the above, wherein the daily prepackaged therapeutic meal is adapted to be provided to a patient selected from the group consisting of: young adults, children and any combination thereof.

It is another object of the current invention to disclose the daily prepackaged therapeutic meal as defined in any of the above, wherein the daily prepackaged therapeutic meal is adapted to be provided to a patient suffering from mild to moderate Ulcerative Colitis.

It is another object of the current invention to disclose the daily prepackaged therapeutic meal as defined in any of the above, wherein the fresh herbs are selected from the group consisting of mint leaves, oregano, rosemary, sage, basil, thyme and any combination thereof.

It is another object of the current invention to disclose the daily prepackaged therapeutic meal as defined in any of the above, wherein the almonds or walnuts are unsalted and unprocessed.

It is another object of the current invention to disclose the daily prepackaged therapeutic meal as defined in any of the above, wherein the food products of stage 1 and stage 2 are fresh.

It is another object of the current invention to disclose the daily prepackaged therapeutic meal as defined in any of the above, wherein the avocado is to be consumed over at least two meals.

It is another object of the current invention to disclose the daily prepackaged therapeutic meal as defined in any of the above, wherein the two peeled potatoes are cooked and refrigerated before consumption.

It is another object of the current invention to disclose the daily prepackaged therapeutic meal as defined in any of the above, wherein the herbal teas may further comprise a slice of fresh lemon, a slice of fresh lime, a slice of fresh orange or mint leaves.

It is another object of the current invention to disclose the daily prepackaged therapeutic meal as defined in any of the above, wherein the food products may be processed in a manner selected from the group consisting of grilling, frying, baking, boiling, broiling, and any combination thereof.

It is another object of the current invention to disclose the daily prepackaged therapeutic meal as defined in any of the above, wherein the food products may be deep fried no more than once a week during stage 1, and no more than twice a week during stage 2.

It is another object of the current invention to disclose the daily prepackaged therapeutic meal as defined in any of the above, wherein a supplemental dose of vitamin D of at least 1,600 IU/day is added to the prepackaged meal.

It is another object of the current invention to disclose the daily prepackaged therapeutic meal as defined in any of the above, wherein stage 1 and stage 2 additionally comprise the step of not consuming the following foods: red meat, pork, turkey, processed meat, seafood, fish, smoked meats and fish, all dairy products except unflavored yoghurt 3-4% fat, non-diary milk products, breads and baked goods of any kind, gluten free products not listed above, potato or corn flour, and any flour not listed as allowed, frozen potatoes or yams, corn during stage 1, legumes, comprising beans, chickpeas and soybeans during stage 1 and white beans during stage 2, margarine, any cooking oils except olive and canola oil, non-dairy creamers or cream, coconut oil, butter, dried fruits, canned fruits, all fruits not on the allowed list, all vegetables not on the allowed list, walnuts from mixes or trail mix, all nuts except unprocessed walnuts and almonds, soft drinks, fruit juices and sweetened beverages, alcoholic beverages, coffee, sauces, spreads, salad dressing, syrups and jams of any kind, canned products, packaged snacks, candy, chocolate, cakes, cookies, chewing gum, artificial sweeteners, mixed spices, ice-cream, yeast for baking.

### BRIEF DESCRIPTION OF FIGURES

FIG. 1: Illustration of the Host-Microbiome Interaction.

FIG. 2: Schematic flow-chart of the Ulcerative Colitis Diet Study Design.

FIG. 3: Schematic flow-chart of the Ulcerative Colitis Diet Study Follow-up schedule.

FIG. 4: Illustration of the main components of the Ulcerative Colitis Diet

FIG. 5: Shows % remission or response to Ulcerative Colitis Diet in a preliminary study

### DETAILED DESCRIPTION OF THE INVENTION

The essence of the present invention is to provide a method and a composition of a diet for an Ulcerative Colitis patient. This diet is designed to reduce exposure to dietary components shown to induce inflammation. The diet of the present invention is designed to avoid consumption of dietary products which may cause dysbiosis or impair the mucous layer or gut barrier, coupled with dietary products which promote gut health and beneficial bacteria.

Dietary products that may cause dysbiosis or impairment of the mucous layer include animal fat, emulsifiers, sulfites, gluten, yeast, and artificial sweeteners such as aspartame and sucralose. Many animal proteins including dairy, beef and fish derived proteins contain high levels of taurine (a sulphonic acid), leading to formation of taurine-conjugated bile acids, both substrates for SRB, and thus, might aggravate ulcerative colitis, and should be avoided.

High sugar and high fat diets may also cause dysbiosis, reduce mucous expression, and increase intestinal permeability. However, certain starch containing carbohydrates and fruits are beneficial since they contain resistant starches, which can be turned into short chain fatty acids. Cooked and refrigerated potatoes and rice are good sources of such carbohydrates. Apple Pectin in the diet may be metabolized by beneficial bacteria such as Fecalibacterium prausnitzii. Processed food is prohibited since manufacturers add sulfites to processed food as preservatives and to improve its appearance. Sulfites can be found in a variety of processed foods, such as cheese, pickles, dried soup mixes, jams, jellies, canned seafood, pie-crusts, dried fruit and many others. Sulfites provide substrates for SRB, which, as stated hereinabove, are deleterious to the microbiome. Artificial sweeteners and maltodextrins may impair goblet cells and promote adherent gut bacteria.

The diet includes mandatory sources of resistant starch such as cooked and refrigerated potatoes, and apples to provide daily apple pectin, as well as fibers from fruits and vegetables as substrates for short chain fatty acid production such as acetate and butyrate.

The term “Ulcerative Colitis (UC)” refers hereinafter to a chronic inflammatory disease primarily involving the colon.

The term “portion” refers hereinafter to a normal serving size of a child, young adult or an adult. More specifically, the term relates to the amount of 150-300 grams.

The term “about” refers hereinafter to ±25% of the defined measure or value.

The term “children” refers hereinafter to a human being at the age range of 8-12.

The term “young adults” refers hereinafter to a human being at the age range of 12-19

The term “Pediatric Ulcerative Colitis Activity Index (PUCAI)” refers hereinafter to a research tool used to quantify the symptoms of patients with Ulcerative Colitis. This tool is used to define remission or response to a treatment. Remission of UC is defined as PUCAI less than 10 at week 6.

The term “endoscopic Mayo Score” refers hereinafter to a tool used to evaluate the UC stage, based on endoscopic exploration. It is a subscore of the Full Mayo Score, and can be used on its own. The scale of the endoscopic Mayo Score: Mayo 0—normal mucosa or inactive disease; Mayo 1—mild activity; Mayo 2—moderate activity; Mayo 3—severe activity.

The term “C-reactive protein (CRP)” refers hereinafter to a protein found in the blood plasma, which levels increase in response to inflammation.

The term “calprotectin” refers hereinafter to a protein found in the stool. Elevated calprotectin levels in the stool, are indicative of bowel inflammation. In Ulcerative colitis patients it associated with disease activity and severity.

The term “fresh food” refers hereinafter to any food that was prepared less than 12 hours in advance, or whole fruits and vegetables that have been picked by have not been processed, altered, canned or have added sweeteners. In order for food to be considered fresh, it cannot be frozen and thawed. This excludes fresh chicken breast, that is considered fresh even if frozen.

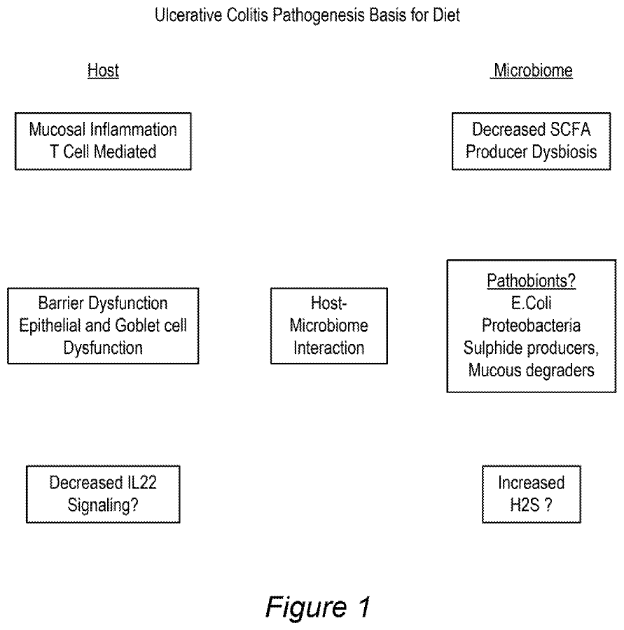

The term “freshly squeezed orange juice” refers hereinafter to orange juice that was squeezed less than an hour before consumption, without addition of chemicals and sweeteners. Orange juice from cartons or bottles is not considered fresh orange juice.
The preliminary data for the UCD are derived from 3 sources:
   1. Preliminary uncontrolled experience in patients refractory to 5ASA (retrospective previously shared)
   2. Prospective pediatric pilot study in 24 children and adolescents refractory to 5ASA
   3. A randomized prospective controlled trial in aults refractory to medication (50% biologic refractory) with documented Mayo2-3 inflammation in which the UCD was one arm of the trial
Adult prospective randomized controlled trial (CRAFT UC)
The Conditioning of Recipient And Donor with Fecal Transplantation in Ulcerative Colitis: (CRAFT UC) trial (NCT 02734589) was a randomized controlled pilot trial involving three arms in adults with refractory established Ulcerative Colitis, over 20 weeks. Patients with active symptomatic disease and a colonoscopy demonstrating moderate to severe endoscopic disease with a Mayo endoscopic score of 2 or 3 were randomized 1:1:1 to one of three groups; Group 1 were allowed to consume a free diet and received a standard FT by colonoscopy on day 1 and rectal enemas from the same donor on days 2 and 14 without dietary conditioning of the donor. Group 2 received FT by colonoscopy on day land rectal enemas from the same donor on days 2 and 14 with dietary pre-conditioning of the donor for 14 days and dietary treatment with a diet (UC Diet) after transplantation and for the following 12 weeks. Group 3 received the UC Diet alone without FT for 12 weeks (FIG. 1).
In this study 15 patients aged mean age 33.8 years (20% failing steroids, 33% biologic refractory) received the UCD for 8 weeks.
Results: Intention to treat steroid free remission was 40% (6/15). The median Simple clinical colitis activity index decreased significantly from 6 (IQR 5-8) to 2 (IQR 0-6) in group 3 (P=0.02). Endoscopic remission occurred in 4/15 (27%) and complete mucosal healing Mayo 0 occurred only with the diet arm in 3/15 (p=0.022). Diet was better than both fecal transplantation arms and the trial was halted due to the better results with diet over fecal transplantation.

### EXAMPLES

### Example 1

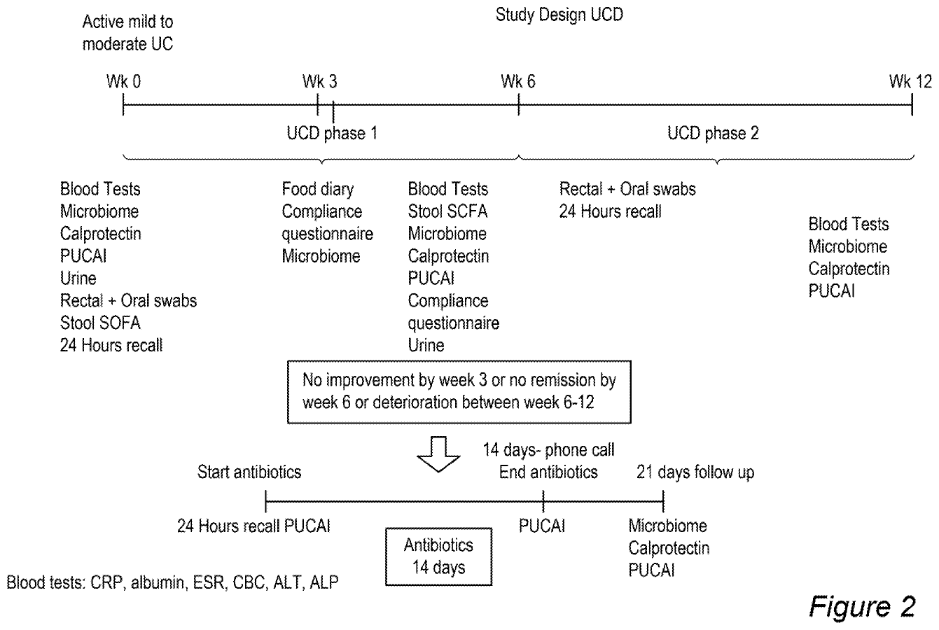

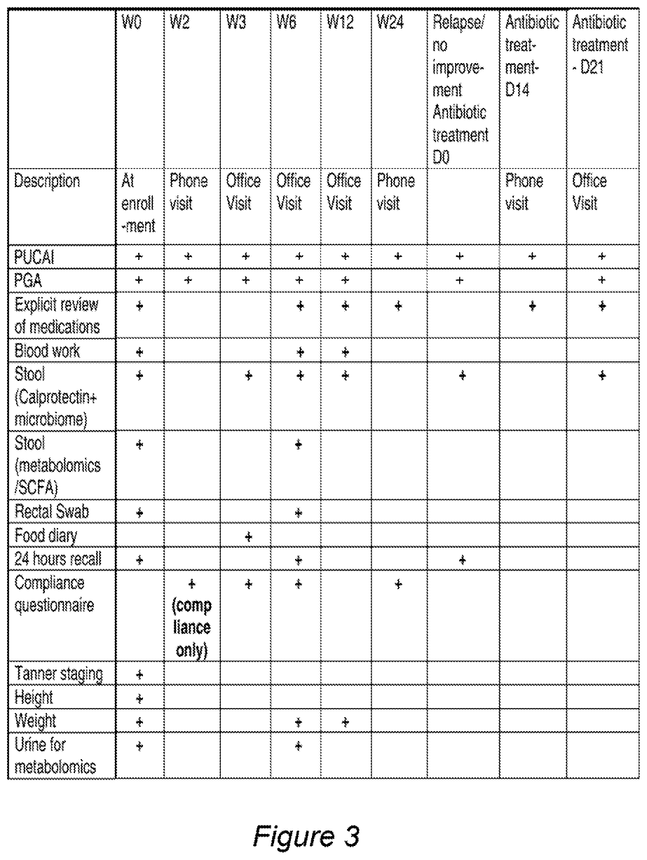

In this example, an open label single arm multicenter pilot study involving treatment of children with active mild to moderate UC using a novel diet with a crossover design for failures is described. All patients meeting inclusion exclusion criteria use a dietary intervention termed the Ulcerative Colitis Diet (UCD), fully described in the following Example 2. The study population comprises 20 UC patients, aged 8-19, with mild to moderate active disease defined by the Pediatric UC Activity Index (10≤PUCAI≤45), at diagnosis, or despite stable maintenance therapy with 5ASA or thiopurines for at least 6 weeks. Patients receive the structured UCD for 6 weeks, and those in remission at week 6 receive the step down diet for another 6 weeks. A timeline summarizing the UCD study design is shown schematically in FIG. 2. Patients are seen at baseline and weeks 3, 6 and 12. At week 2 a phone call visit to assess PUCAI and dietary compliance is also performed. At each visit a PUCAI is recorded. Standard blood results (CRP, albumin, ESR, CBC, ALT, ALP) are be performed at weeks 0, 6 12 weeks. Stool samples for calprotectin are collected at weeks 0, 3, 6 and 12. A food diary is collected at week 3. A compliance questionnaire will be completed on weeks 3, 6. A 24 hours recall will be collected at weeks 0, 6. The cohort will be followed for 24 weeks (12 weeks of study participation, with an additional 12 weeks of chart review and a telephone call at 24 weeks). A table summarizing patient follow-up is shown in FIG. 3. The follow up phase will consist of extraction of data from the medical chart and a telephone call asking general questions regarding continued adherence to UCD.

Performance of sigmoidoscopy or colonoscopy is not part of the protocol, but if one is performed during the 12 week period or in the ensuing month after therapy as standard of care for assessment of response, participants are requested to register the partial endoscopic Mayo score in each segment (rectum, sigmoid, descending colon etc.), using the highest score in each segment. Since the diet does restrict many forms of calcium, patients will need to take calcium supplementation to meet basic dietary requirements while on the UCD.

Dietary Intervention

All patients receive an in identical dietary format, to be used for a period of six weeks, and then a step down diet for an additional six week period. A detailed description of the diet is given in Example 2. The first period involves a more restricted diet. All patients receive a handout with instructions regarding the diet, and preparation of food, including a list of allowed and disallowed food and products. An explanation regarding the principles is to be given to all patients by a dietician or one of the physicians.

Inclusion Criteria:

1. Informed consent

2. Established diagnosis of UC.

3. Age: 8-19 years (inclusive)

4. Mild to moderate active disease, 10≤PUCAI≤45.

5. Stable medication (IMM/5ASA) use or no medication use for the past 6 weeks. Patients who have received topical 5ASA therapy for <7 days and are active may be included if topical therapy is stopped at enrolment.

Exclusion Criteria:

1. Any proven current infection such as positive stool culture, parasite or positive C. diffcile toxin.

2. Antibiotic or Steroids use in the past 2 weeks.

3. Use of biologics in present or in past

4. PUCAI>45

5. Acute severe UC (PUCAI>60) in the previous 12 months.

6. Current Extra intestinal manifestation of UC.

7. PSC or Liver disease

8. Pregnancy.

9. Allergy to one of the antibiotics or age <11 will exclude patients from entering the antibiotic arm

10. Fever >38 degrees centigrade.

Exclusion Criteria Comments:
1. Stool culture, parasite or C. difficile toxin is only measured if the patient has diarrhea.
2. Patients who have received treatment enemas for up to 6 days, and are active, must stop the enemas at enrolment.

Translational Aspects:

A stool sample for calprotectin and analysis of the microbiota (16SRNA) will also be collected at weeks 0, 3, 6 & 12. Stool samples for Metabolomics and Short Chain Fatty Acid analysis will be collected at weeks 0 and 6. A rectal and oral swab for mucosal adjacent bacterial analysis is to be performed at weeks 0 and 6; this test is optional and not obligatory for recruitment. Urine samples for metabolomics will be collected at baseline and week 6.

Stool DNA will be extracted and sequenced using next-generation sequencing technology to characterize the microbiome of trial UC patients before, during, and after dietary therapy. Our primary analytical aim is to integrate functional profiles of microbial communities and metagenome-based assessment of community structure/function with patient metadata. Profiles of community function will be inferred from 16S ribosomal RNA (rRNA) gene sequencing data using the functional prediction software Phylogenetic Investigation of Communities by Reconstruction of Unobserved States (PICRUSt). Whole metagenome sequencing may also be performed, in order to study the collective functional potential of each sample microbiome. A novel modeling-framework called BiomeNet will be used to discriminate groups of samples according to metabolic patterns discovered within the gut microbiome. Results will take the form of hierarchical systems of enzyme-mediated metabolic reactions. BiomeNet will be used for the following: (i) to identify dependencies between gut metabolic phenotypes and response to dietary therapy, and (ii) to determine if any of those metabolic phenotypes differ according to the type of therapy (CDED+EN vs, EEN). A second modelling framework called BioMico will be used to learn the components of community structure (as inferred from 16S sequence data) that have the most power to discriminate samples according to the labeled features of their metadata [2]. The model will be trained according to the clinical data parameters collected during this study (e.g. disease activity, disease phenotype, patient characteristics). The accuracy of using microbial community structures to predict patient phenotypes will be quantified via a rigorous method of statistical cross-validation [2, 3]. These analyses of community structure and metabolic function will be integrated. After using BiomeNet to identify the metabolic subsystems associated with a response to dietary intervention, input of only the key reactions into BioMiCo will be carried out, which will then be trained to discriminate patient groups according to labels derived from the clinical data collected during this study. In this way, explanation of patient phenotypes in terms of the metabolic interactions within their communities may be attempted. Through a combination of analytical approaches (BioMiCo, PICRUSt & BiomeNet), specific functional and phylogenetic markers associated with treatment response will be identified.

Primary End Point

Remission rate, defined as a PUCAI less than 10 at week 6.

Secondary End Points

Mean PUCAI week 6

Mean Calprotectin at week 6

Remission week 12

Physicians Global Assessment week 6

Mean PUCAI week 12

### Example 2

Ulcerative Colitis Diet (UCD)

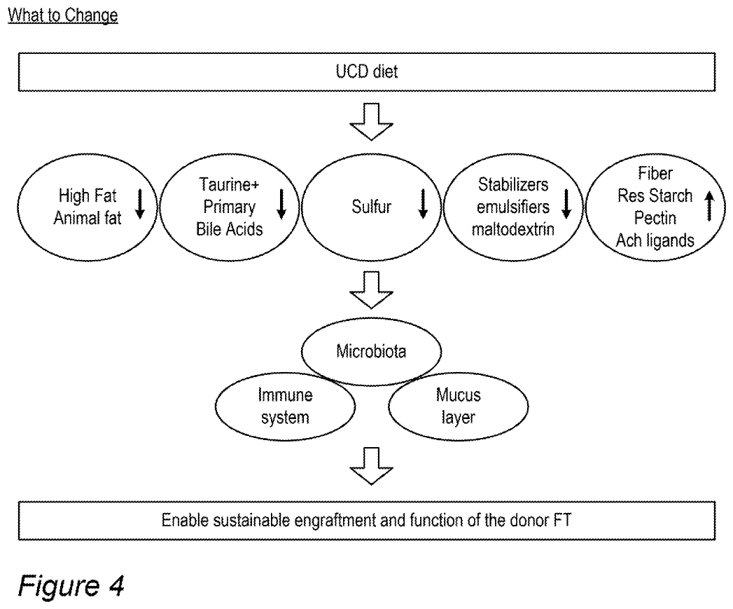

The ulcerative colitis diet is used to reduce inflammation and regain control of the disease. The following diet is used for 12 weeks, and consists of two stages. The first stage diet is used for weeks 0-6, and the second stage diet is used for weeks 7-12. The first stage diet is more restrictive than the second stage diet. An illustration depicting the main components of the diet is shown in FIG. 4.

The diet consists of mandatory foods, allowed foods, and foods that are restricted in quantity of frequency of exposure. The diet is divided into food groups, and each group contains allowed and disallowed foods. Foods that do not appear in the allowed list, are not allowed, even if they are not specifically mentioned in the list of disallowed foods.

Foods may be grilled, barbequed, pan-fried, baked, boiled and broiled. During the first stage (weeks 0-6), food may be deep-fried, up to once a week. During the second stage (weeks 7-12), food be deep-fried up to twice a week.

Vitamin D supplements, at least 1,600 IU a day must be taken every day.

Patients eating out in restaurants, should eat freshly grilled chicken, white rice and baked potatoes. Fried eggs or hard boiled eggs may also be eaten. Scrambled eggs may be made from mixes or contain dairy products, and should be avoided. Restaurants will most likely use prepared sauces, dressings and mixes, and food containing these products should be avoided. Mashed potatoes are allowed, providing they are made from fresh potatoes, and not from dehydrated potatoes, and that no dairy products are added. Frozen potatoes, used for fries in most restaurants must also be avoided.

### Ulcerative Colitis Diet Week 0-6

Meat and Protein

Mandatory:

Fresh chicken breast, portion size per day: 150 gr for patients weighing under 40 kg, and 200 gr for patients weighing over 40 kg, 3 times a week.

1 egg daily (for patients weighing under 40 kg)

2 eggs daily (for patients weighing over 40 kg, and vegetarians)

Allowed with Restrictions:

Tofu—200 gr once a week from week 4

3 additional eggs a week for cooking or baking

Plain unflavored regular (3-4%) fat yoghurt, up to 3 times a week.

Disallowed Meat and Protein:

Red meat, pork, turkey, processed meat, seafood and fish, smoked meats and fish, sausages, luncheon meats, salamis, fish sticks, smoked salmon, canned fish, dairy products other than plain yoghurt, milk beverages (soy mild, rice milk, almond milk etc.)

Carbohydrates:

Mandatory:

2 fresh peeled potatoes daily (cooked and refrigerated before use)

Allowed—Unlimited:

White rice

Rice noodles or rice papers

Rice flour

Allowed with Restrictions:

Oatmeal, up to ¼ cup (dry) a day

Pasta: durum pasta, up to 200 grams

Legumes: lentils/peas/cooked green beans, up to 4 tablespoons a day

Quinoa: up to 4 tablespoons cooked quinoa a day

Disallowed Carbohydrates:

Bread, store bought baked goods of any kind, yeast, gluten-free products not listed above, potato or corn flour, other flours not listed as allowed, corn, frozen potatoes, legumes: beans, chickpeas and soybeans.

Oils

Allowed:

Olive oil and canola oil

Disallowed:

Other cooking oils not listed above, margarine, non-dairy creamers or cream, coconut oil, butter

Fresh Fruits

Mandatory:

2 bananas per day,

1 apple per day

Allowed with Restrictions:

1 slice melon (cantaloupe, honeydew or watermelon) per day

Fresh berries: 5 strawberries or 5 raspberries or 10 blueberries per day

Choice of: 1 kiwi or 10 seedless grapes or 1 pear, per day

Disallowed:

Dried fruit, and all other fruits not on the allowed list

Fresh Vegetables:

Mandatory:

At least one portion of fresh salad (lettuce or spinach leaves with vegetables or mixed vegetable salad) every day from foods listed as allowed in the vegetable section below, or from the allowed fruit section above.

Allowed with Restrictions, (Daily):

2 tomatoes, 2 cucumbers, 3 lettuce leaves 1 carrot, 1 zucchini, 3 broccoli or cauliflower florets or clusters, 1 avocado (not more than ½ an avocado per meal), 100 gr pumpkin, ½ red bell pepper.

Disallowed:

Cabbage, kohlrabi, brussels sprouts, leeks, radish onion and all other vegetables not on the allowed list.

Beverages

Allowed:

Water, soda, herbal teas (a slice of fresh lemon, a slice of fresh lime, a slice of orange or mint leaves may be added to tea for taste), chamomile tea, one glass of freshly squeezed orange juice per day (not from cartons or bottles)

Disallowed:

All soft drinks, diet beverages, fruit juices and sweetened beverages, alcoholic beverages, coffee.

Nuts

Allowed with Restrictions:

6 unprocessed walnut halve or unprocessed unroasted almonds—may be consumed daily only after diarrhea and bleeding have stopped.

Disallowed:

Roasted walnuts, walnuts from mixes or trail mix, all other nuts.

Beverages

Allowed:

Water, soda, herbal teas (a slice of fresh lemon, a slice of fresh lime, a slice of orange or mint leaves may be added to tea for taste), chamomile tea, one glass of freshly squeezed orange juice per day (not from cartons or bottles)

Disallowed:

All soft drinks, diet beverages, fruit juices and sweetened beverages, alcoholic beverages, coffee.

Condiments for Cooking

Allowed:

Spices (pure): salt, pepper, paprika, cinnamon, curcumin, turmeric.

Fresh herbs: mint leaves, oregano, coriander, rosemary, sage, basil, thyme.

Fresh ginger, fresh lemon juice, fresh tomato sauce, baking soda.

Allowed with Restrictions:

Pure honey for cooking or desserts, up to 3 tablespoons daily

Table sugar, up to 3 teaspoons per day, for cooking or tea

Disallowed:

Sauces, spreads, salad dressings, syrups and jams of any kind, canned products, packaged snacks (e.g. potato chips, pretzels, popcorn, nuts), candy, chocolate, cake, cookies, chewing gum, artificial sweeteners, mixed spices, ice-cream.

### Ulcerative Colitis Diet Week 7-12

Meat and Protein

The same as weeks 0-6, with the addition of plain yoghurt being allowed every day, and an additional 1 egg (up to two a day).

Carbohydrates

The same as weeks 0-6 with the addition of:

One of the 2 mandatory fresh peeled potatoes may be replaced by one sweet potato,

Oatmeal—up to ½ cup per day

1 additional sweet potato

Legumes; dried chickpeas may be added as a legume

I slice of whole grain bread or ½ cup bread crumbs, daily

Corn—1 cob per day

Oils

The same as weeks 0-6

Fresh Fruits

Mandatory:

1 banana

1 apple

1 additional fruit from allowed list

Allowed with Restrictions:

1 slice melon (honeydew, cantaloupe) daily

Fresh berries: 5 strawberries or 5 raspberries or 10 blueberries, per day

Choice of: 1 kiwi or 10 seedless grapes or 1 pear or 4 cubes of mango or 1 slice pineapple or 1 tangerine or ½ an orange, per day.

Fresh Vegetables

The same as weeks 0-6, with the addition of:

½ a red/yellow/green bell pepper (instead of only ½ a red pepper) per day

4 fresh button mushrooms.

Nuts

The same as weeks 0-6.

Beverages

The same as weeks 0-6, with the addition of: any home-made juice smoothie.

Condiments for Cooking

The same as weeks 0-6, with the addition of:

Unsweetened cocoa powder without additives and sulfite free raisins for baking

### Example 3

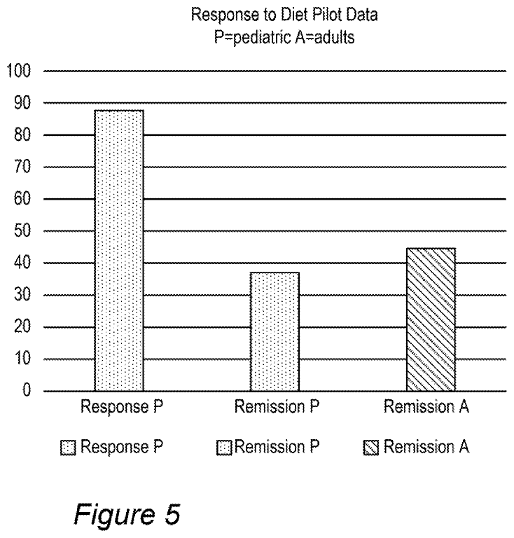

A pilot study in 9 patients with mild to moderate UC was carried out in order to evaluate the benefit of the Ulcerative Colitis Diet (UCD) protocol before launching an extensive study. Eight out of nine had improved symptoms with decreased diarrhea and bleeding, 5/9 (55%) patients were in remission by 6 weeks. The data obtained from the pilot study, is summarized in FIG. 5.

Patient #1: Remision.

A 16 year old female with a PUCAI of 35, contiguous disease until 60 cm, Mayo 2 endoscopic score throughout. She was in remission by week 6 (PUCAI 0), and continued to be in remission until week 14 (PUCAI 0 at week 12), at which time, she relapsed (virus?). Her sigmoidoscopy revealed colitis endoscopic mayo score 3 in the rectosigmoid until 15 cm, and then Mayo 2 until 90 cm. She received the antibiotic rescue and went into complete remission, her PUCAI at day 21 was 0. She has been in remission for 3 months since stopping antibiotics while continuing the diet.

Patient #2—Response.

A 15 year old female PUCAI 25, who relapsed every time she stopped daily enemas with oral 5 ASA. She stopped enemas and relapsed. She was in complete remission by week 3 (PUCAI 5) but relapsed at the week 6 visit (PUCAI 15), and commenced 5ASA enemas by week 6 and improved with the enemas. She discontinued the diet.

Patient #3—Remission

An 11 year old female with active colitis to 60 cm, until 50 cm Mayo 2 and from 50-60 cm Mayo 1. Her PUCAI was 45. At week 6 she was in remission with PUCAI 0, which continued to week 12. A repeat sigmoidoscopy one month later revealed normal mucosa until 40 cm, a 10 cm patch of colitis Mayo 2, and normal mucosa until 60 cm. She ceased the diet and relapsed, returned to the stage 1 diet and is still in remission 4 months later, on a partial elimination diet.

Patient #4—Remission.

A 32 year old male with left sided colitis despite 4 grams 5ASA and 1 gr 5 ASA enemas, was not in remission for close to a year. He refused steroids and approached us for dietary therapy. His PUCAI was 20, he went in to remission with a PUCAI 5, and came back for only follow up, though he self-reported sustained remission after that by telephone follow up at week 12 and at week 16. He had a mild relapse after stopping the diet and then returned to first stage diet and went in to remission.

Patient #5—Remission.

A 16 year old male, on oral mesalamine, started with a PUCAI 30, went in to remission by week 3 PUCAI 0, but did not return for follow up (change in insurance coverage). A telephone call after 6 weeks indicated clinical remission. He is lost to follow up.

Patients #6—Remission

A 31 year old female was diagnosed 4 months previously with left sided ulcerative colitis. She was started with 4 grams 5ASA and enemas, but did not enter remission, and flared while on the therapy. She was started on 40 mg prednisone with only a mild response and continued to have 3-4 diarrhea episodes with bleeding daily. She started the diet and went in to remission, steroids were tapered and she remained in remission for two years. She had a severe flare after two years after stopping the diet and was hospitalized for steroid therapy with over 12 bloody stools a day, abdominal pain and nocturnal diarrhea. She refused steroids. She restarted the diet and was in remission in 6 weeks, a subsequent colonoscopy showed only very mild inflammation (Mayo 1).

Patient #7—Response.

An 18 year old female, her initial PUCAI at week 0 was 45, at week 2 it was 15, and continued to have mild disease until week 6 (PUCAI 15). She received the antibiotics, which did not lead to remission, her PUCAI remained 15.

Patient #8—Response.

A 28 year old female, with failed oral mesalame and topical 5ASA. Her PUCAI was 40. She modified the diet and adhered to the diet only when at home, did not adhere to the diet when she was away from home. She also stopped all the medications while using the diet. She reported significant improvement, her PUCAI declined to 25 at week 6, and has discontinued all drugs during the diet, at her own discretion.

Patient #9—Failure.

A 19 year old female, her PUCAI was 50, but increased to 65 on the day of the diet. The PUCAI was 55 at week 3 and she discontinued the diet.

### REFERENCES

1. Jostins J, Ripke S. Weersma R et al. Host-microbe interactions have shaped the genetic architecture of inflammatory bowel disease. Nature 2012; 491:119-24.
2. Manichanh C, Borruel N, Casellas F et al. The gut microbiota in IBD. Nat Rev Gastroenterol Hepatol. 2012; 9:599-608.
3. Noor S O, Ridgway K, Scovell L, et al. Ulcerative colitis and irritable bowel patients exhibit distinct abnormalities of the gut microbiota. BMC Gastroenterol 2010; 10:134.
4. Davenport M, Poles J, Leung J M et al. Metabolic alterations to the mucosal microbiota in inflammatory bowel disease. Inflamm Bowel Dis. 2014; 20:723-31.
5. David L A, Maurice C F, Carmody R N, et al. Diet rapidly and reproducibly alters the human gut microbiome. Nature. 2014; 505:559-63.
6. James S L, Christophersen C T, Bird A R, et al. Abnormal fibre usage in UC in remission.

Gut. 2014, 18: gutjnl-2014-307198.
7. Khalil N A, Walton G E, Gibson G R et al. In vitro batch cultures of gut microbiota from healthy and ulcerative colitis (UC) subjects suggest that sulphate-reducing bacteria levels are raised in UC and by a protein-rich diet. Int J Food Sci Nutr. 2014; 65:79-88.
8. Johansson M E, Gustafsson J K, Holmen-Larsson J et al. Bacteria penetrate the normally impenetrable inner colon mucus layer in both murine colitis models and patients with ulcerative colitis. Gut. 2014; 63:281-91.
9. De Preter V, Arijs I, Windey K et al. Decreased mucosal sulfide detoxification is related to an impaired butyrate oxidation in ulcerative colitis. Inflamm Bowel Dis. 2012; 18:2371-80.
10. Jowett S L, Seal C J, Pearce M S et al. Influence of dietary factors on the clinical course of ulcerative colitis. Gut 2004; 53:1479-1484.
11. Pitcher M C L et al, The contribution of sulphate reducing bacteria and 5-aminosalicylic acid to faecal sulphide in patients with ulcerative colitis, Gut 2000; 46:64-72.
12. Devkota S, Wang Y, Musch M, et-al. Dietary-fat-induced taurocholic acid promotes pathobiont expansion and colitis in IL10−/− mice. Nature 2012; 487: 104-9.
13. Fuentes S, Rossen N G, Van der Spek M J et al. Microbial shifts and signatures of long-term remission in ulcerative colitis after faecal microbiota transplantation. ISME J. 2017 August; 11(8):1877-1889 F
14. Ohkusa T, Kato K, Terao S, Chiba T, Mabe K, Murakami K, et al. Newly Developed Antibiotic Combination Therapy for Ulcerative Colitis: A Double-Blind Placebo-Controlled Multicenter Trial. Am J Gastroenterol. 2010; 105:1820-1829.
15. Turner D, Levine A, Kolho K L et al. Combination of oral antibiotics may be effective in severe pediatric ulcerative colitis: A preliminary report. J Crohns Colitis. 2014; 8:1464-70.
16. Terao S, Yamashiro K, Tamura I et al. Antibiotic combination therapy for steroid withdrawal in steroid-dependent ulcerative colitis. Digestion. 2011; 83:198-203.
17. Kato K, Ohkusa T, Terao S et al. Adjunct antibiotic combination therapy for steroid-refractory or -dependent ulcerative colitis: an open-label multicentre study. Aliment Pharmacol Ther. 2014; 39:949-56.
18. Albenberg L G, Wu G D. Diet and the intestinal microbiome: associations, functions, and implications for health and disease. Gastroenterology. 2014; 146:1564-72.
19. Wedlake L, Slack N, Jervoise N H et al. Fiber in the Treatment and Maintenance of Inflammatory Bowel Disease: A systematic Review of randomized controlled trials, Inflamm Bowel Dis 2014; 20:576-586.
20. Kyaw M H, Moshkovska T, Mayberry J. A prospective, randomized, controlled, exploratory study of comprehensive dietary advice in ulcerative colitis: impact on disease activity and quality of life. Eur J Gastroenterol Hepatol. 2014; 26:910-7.
21. Hafer A, Kramer S, Duncker S et al. Effect of oral lactulose on clinical and immunohistochemical parameters in patients with inflammatory bowel disease: a pilot study. BMC Gastroenterol. 2007 Sep. 4; 7:36

1.-52. (canceled)

53. A method for providing a two stage diet for an Ulcerative Colitis patient, said method comprising stage 1 which is provided in weeks 0-6, and stage 2 which is provided in weeks 7-12;
wherein stage 1 comprises the step of consuming the following food products;
(a) at least one portion of fresh chicken breast three times a week, two peeled potatoes a day, 2 bananas a day, 1 apple a day, at least one portion of fresh salad a day from spinach or lettuce leaves with daily allowed fruit or vegetables, one of the following: one egg a day for patients below 40 kg, or two eggs a day for vegetarian patients and patients above 40 kg;
(b) unlimited amounts of products selected from the group consisting of: white rice, rice noodles/rice papers, rice flour, olive oil, canola oil, salt, pepper, paprika, cinnamon, curcumin, turmeric, fresh herbs, fresh ginger, fresh lemon juice, fresh tomato sauce, baking soda, water, soda, herbal teas, chamomile tea;
(c) no more by quantity than the following: tofu, 200 grams once a week from week 4, 3 additional eggs a week for cooking or baking, plain unflavored regular (3-4%) fat yoghurt 3 times a week, oatmeal up to ¼ cup (dry) a day, almond flour, up to ¼ cup weekly, cooked durum pasta, up to 200 grams, twice a week, cooked quinoa, up to 4 tablespoons a day, legumes, selected from the group consisting of: lentils, peas or cooked green beans, up to 4 tablespoons a day, fresh melon selected from the group consisting of cantaloupe, honeydew or watermelon, up to one slice daily, fresh berries daily selected from the group consisting of five strawberries, five raspberries or ten blueberries, one fresh fruit per day, selected from the group consisting of: 1 kiwi, 10 seedless grapes or 1 pear, 2 tomatoes daily, 2 cucumbers daily, 3 lettuce leaves daily, 1 cup spinach leaves daily, 1 carrot daily, 1 zucchini daily, 3 broccoli or cauliflower florets or clusters daily, 1 avocado daily, 100 grams pumpkin daily, ½ red pepper daily, 6 unprocessed walnut halves or unprocessed unroasted almonds a day, one glass of freshly squeezed orange juice per day, pure honey for cooking or desserts, up to 3 tablespoons a day and table sugar for cooking or tea, up to 3 teaspoons a day;
further wherein stage 2 comprises the step of consuming the following food products:
(a) one portion of fresh chicken breast 3 times a week, one of the following: one egg a day for patients below 40 kg, or two eggs a day for vegetarian patients and patients above 40 kg, 2 fresh peeled potatoes or 1 peeled potato and 1 peeled sweet potato, daily, 1 banana, 1 apple (not cooked), and one additional fruit daily, selected from the group consisting of: 1 slice melon (cantaloupe honeydew), fresh berries daily, selected from the group consisting of: 5 strawberries, 5 raspberries or 10 blueberries and one portion of salad daily, comprising a combination of allowed vegetables, fruits and nuts;
(b) unlimited amounts of products selected from the group consisting of: white rice, rice noodles/rice papers, rice flour, lettuce/roquette, arugula, olive oil, canola oil, water, soda, herbal teas, chamomile tea, any home-made juice smoothie, salt, pepper, paprika, cinnamon stick, curcumin, turmeric, fresh herbs, fresh ginger, fresh lemon juice, fresh tomato sauce, unsweetened cocoa powder without additives and sulfite-free dark raisins for baking;
(c) no more by quantity than the following: Tofu 200 grams, once a week, 1 plain unflavored regular (3-4%) fat yoghurt daily, 1 additional egg a day, oatmeal, up to ½ a cup per day, wheat or spelt flour for baking, up to ½ cup once a week, 1 additional sweet potato daily, cooked quinoa, up to 4 tablespoons a day, legumes, selected from the group consisting of: lentils, peas, green beans, or dried chickpeas, up to 5 tablespoons a day, 1 slice of whole grain bread or ½ a cup of bread crumbs daily, cooked durum pasta, 200 grams, up to 2 times a week, one corn cob a day, fresh melon selected from the group consisting of cantaloupe, honeydew or watermelon, up to one slice daily, fresh berries selected from the group consisting of five strawberries, five raspberries or ten blueberries, a day, one fresh fruit a day, selected from the group consisting of: 1 kiwi, 10 seedless grapes or 1 pear, 4 cubes of mango, 1 slice pineapple, 1 tangerine or one orange, 2 tomatoes daily, 2 cucumbers daily, 1 carrot daily, 1 zucchini daily, 3 broccoli or cauliflower florets or clusters daily, 1 avocado daily, 100 grams pumpkin, 2 red, yellow or green bell pepper a day, 4 fresh button mushrooms a day, 6 walnut halves or 6 almonds a day, one glass of freshly squeezed orange juice per day, pure honey for cooking, up to 3 tablespoons a day and sugar for tea or cooking, up to 3 teaspoons a day;
thereby providing dietary components comprising food with reduced content of animal fat, dairy products, gluten, taurine, yeast, sulfites, and emsulsifiers.

54. The method of claim 53, wherein said patient is suffering from mild to moderate Ulcerative Colitis.

55. The method of claim 53, wherein said food products of said stage 1 and stage 2 are fresh.

56. The method of claim 53, wherein said food products may be processed in a manner selected from the group consisting of grilling, frying, baking, boiling, broiling, and any combination thereof.

57. The method of claim 53, wherein said stage 1 and said stage 2 additionally comprise the step of not consuming the following foods: red meat, pork, turkey, processed meat, seafood, fish, smoked meats and fish, all dairy products except unflavored yoghurt 3-4% fat, non-dairy milk products, breads and baked goods of any kind, gluten free products not listed above, potato or corn flour, and any flour not listed as allowed, frozen potatoes or yams, corn during stage 1, legumes, comprising beans, chickpeas and soybeans during stage 1 and white beans during stage 2, margarine, any cooking oils except olive and canola oil, non-dairy creamers or cream, coconut oil, butter, dried fruits, canned fruits, all fruits not on the allowed list, all vegetables not on the allowed list, walnuts from mixes or trail mix, all nuts except unprocessed walnuts and almonds, soft drinks, fruit juices and sweetened beverages, alcoholic beverages, coffee, sauces, spreads, salad dressing, syrups and jams of any kind, canned products, packaged snacks, candy, chocolate, cakes, cookies, chewing gum, artificial sweeteners, mixed spices, ice-cream and yeast for baking.

58. A range of food products for an Ulcerative Colitis remission diet; said range of food products comprise a first range of food products and a second range of food products; said first range of food products is adapted to be consumed during weeks 0-6 of said diet, and second range of products is adapted to be consumed in weeks 7-12;
wherein said first range of food products comprise the following food products:
(a) at least one portion of fresh chicken breast three times a week, two peeled potatoes a day, 2 bananas a day, 1 apple a day, at least one portion of fresh salad a day from spinach or lettuce leaves with daily allowed fruit or vegetables, one of the following: one egg a day for patients below 40 kg, or two eggs a day for vegetarian patients and patients above 40 kg;
(b) unlimited amounts of products selected from the group consisting of: white rice, rice noodles/rice papers, rice flour, olive oil, canola oil, salt, pepper, paprika, cinnamon, curcumin, turmeric, fresh herbs, fresh ginger, fresh lemon juice, fresh tomato sauce, baking soda, water, soda, herbal teas, chamomile tea;
(c) no more by quantity than the following: tofu, 200 grams once a week from week 4, 3 additional eggs for cooking or baking per week, plain unflavored regular (3-4%) fat yoghurt 3 times a week, oatmeal up to ¼ cup (dry) a day, almond flour, up to ¼ cup weekly, cooked durum pasta, up to 200 grams, twice a week, cooked quinoa, up to 4 tablespoons a day, legumes, selected from the group consisting of: lentils, peas or cooked green beans, up to 4 tablespoons a day, fresh melon selected from the group consisting of cantaloupe, honeydew or watermelon, up to one slice daily, fresh berries selected from the group consisting of five strawberries, five raspberries or ten blueberries, a day, one fresh fruit a day, selected from the group consisting of: 1 kiwi, 10 seedless grapes or 1 pear, 2 tomatoes daily, 2 cucumbers daily, 3 lettuce leaves daily, 1 cup spinach leaves daily, 1 carrot daily, 1 zucchini daily, 3 broccoli or cauliflower florets or clusters daily, 1 avocado daily, 100 grams pumpkin daily, ½ red pepper daily, 6 unprocessed walnut halves or unprocessed unroasted almonds a day, one glass of freshly squeezed orange juice per day, pure honey for cooking or desserts, up to 3 tablespoons a day and table sugar for cooking or tea, up to 3 teaspoons a day;
further wherein said second range of food products comprise the following food products:
(a) one portion of fresh chicken breast 3 times a week, one of the following: one egg a day for patients below 40 kg, or two eggs a day for vegetarian patients and patients above 40 kg, 2 fresh peeled potatoes or 1 potato and 1 sweet potato, daily, 1 banana, 1 apple (not cooked), and one additional fruit daily, selected from the group consisting of: 1 slice melon (cantaloupe honeydew), fresh berries daily, selected from the group consisting of: 5 strawberries, 5 raspberries or 10 blueberries, one portion of salad daily, comprising a combination of allowed vegetables, fruits and nuts;
(b) unlimited amounts of products selected from the group consisting of: white rice, rice noodles/rice papers, rice flour, lettuce/roquette, arugula, olive oil, canola oil, water, soda, herbal teas, chamomile tea, any home-made juice smoothie, salt, pepper, paprika, cinnamon stick, curcumin, turmeric, fresh herbs, fresh ginger, fresh lemon juice, fresh tomato sauce, unsweetened cocoa powder without additives, sulfite-free dark raisins for baking;
(c) no more by quantity than the following: Tofu 200 grams, once a week, 1 plain unflavored regular (3-4%) fat yoghurt daily, 1 additional egg, oatmeal, up to ½ a cup per day, wheat or spelt flour for baking, up to ½ cup once a week, 1 additional sweet potato daily, cooked quinoa, up to 4 tablespoons a day, legumes, selected from the group consisting of: lentils, peas, green beans, or dried chickpeas, up to 5 tablespoons a day, 1 slice of whole grain bread, or ½ a cup of bread crumbs daily, cooked durum pasta, 200 grams, up to 2 times a week, one corn cob a day, fresh melon selected from the group consisting of cantaloupe, honeydew or watermelon, up to one slice daily, fresh berries daily, selected from the group consisting of five strawberries, five raspberries or ten blueberries, one fresh fruit a day, selected from the group consisting of: 1 kiwi, 10 seedless grapes or 1 pear, 4 cubes of mango, 1 slice pineapple, 1 tangerine or one orange, 2 tomatoes daily, 2 cucumbers daily, 1 carrot daily, 1 zucchini daily, 3 broccoli or cauliflower florets or clusters daily, 1 avocado daily, 100 grams pumpkin, 2 red, yellow or green bell pepper a day, 4 fresh button mushrooms a day, 6 walnut halves or 6 almonds a day; one glass of freshly squeezed orange juice per day, pure honey for cooking or desserts, up to 3 tablespoons a day and table sugar for cooking or tea, up to 3 teaspoons a day;
thereby providing dietary components comprising food with reduced content of animal fat, dairy products, gluten, taurine, yeast, sulfites, and emsulsifiers.

59. The range of food products of claim 53, wherein said range of food is adapted to be provided to a patient suffering from mild to moderate Ulcerative Colitis.

60. The range of foods of claim 53, wherein said food products of said stage 1 and stage 2 are fresh.

61. The range of foods of claim 53, wherein said food products may be processed in a manner selected from the group consisting of grilling, frying, baking, boiling, broiling, and any combination thereof.

62. The range of foods of claim 53, wherein said stage 1 and said stage 2 additionally comprise the step of not consuming the following foods: red meat, pork, turkey, processed meat, seafood, fish, smoked meats and fish, all dairy products except unflavored yoghurt 3-4% fat, non-diary milk products, breads and baked goods of any kind, gluten free products not listed above, potato or corn flour, and any flour not listed as allowed, frozen potatoes or yams, corn during stage 1, legumes, comprising beans, chickpeas and soybeans during stage 1 and white beans during stage 2, margarine, any cooking oils except olive and canola oil, non-dairy creamers or cream, coconut oil, butter, dried fruits, canned fruits, all fruits not on the allowed list, all vegetables not on the allowed list, walnuts from mixes or trail mix, all nuts except unprocessed walnuts and almonds, soft drinks, fruit juices and sweetened beverages, alcoholic beverages, coffee, sauces, spreads, salad dressing, syrups and jams of any kind, canned products, packaged snacks, candy, chocolate, cakes, cookies, chewing gum, artificial sweeteners, mixed spices, ice-cream, yeast for baking.

63. A method of preventing Ulcerative Colitis to be followed for at least 12 weeks, said method comprising the following steps of limiting all food intake to a first range of food products in weeks 0-6 and a second range of food products in weeks 7-12;
wherein said first range of food products comprise the following food products;
(a) at least one portion of fresh chicken breast three times a week, two potatoes a day, 2 bananas a day, 1 apple a day, at least one portion of fresh salad a day from spinach or lettuce leaves with daily allowed fruit or vegetables and one of the following: one egg a day for patients below 40 kg, or two eggs a day for vegetarian patients and patients above 40 kg;
(b) unlimited amounts of products selected from the group consisting of: white rice, rice noodles/rice papers, rice flour, olive oil, canola oil, salt, pepper, paprika, cinnamon, curcumin, turmeric, fresh herbs, fresh ginger, fresh lemon juice, fresh tomato sauce, baking soda, water, soda, herbal teas, chamomile tea;
(c) no more by quantity than the following: tofu, 200 grams once a week from week 4, 3 additional eggs for cooking or baking per week, plain unflavored regular (3-4%) fat yoghurt 3 times a week, oatmeal up to ¼ cup (dry) a day, almond flour, up to ¼ cup weekly, cooked durum pasta, up to 200 grams, twice a week, cooked quinoa, up to 4 tablespoons a day, legumes, selected from the group consisting of: lentils, peas or cooked green beans, up to 4 tablespoons a day, fresh melon selected from the group consisting of cantaloupe, honeydew or watermelon, up to one slice daily, fresh berries selected from the group consisting of five strawberries, five raspberries or ten blueberries a day, one fresh fruit daily, selected from the group consisting of: 1 kiwi, 10 seedless grapes or 1 pear, 2 tomatoes daily, 2 cucumbers daily, 3 lettuce leaves daily, 1 cup spinach leaves daily, 1 carrot daily, 1 zucchini daily, 3 broccoli or cauliflower florets or clusters daily, 1 avocado daily, 100 grams pumpkin daily, 2 red pepper daily, 6 unprocessed walnut halves or unprocessed unroasted almonds a day, one glass of freshly squeezed orange juice per day, pure honey for cooking or desserts, up to 3 tablespoons a day and table sugar for cooking or tea up to 3 teaspoons a day;
further wherein said second range of food products comprise the following food products:
(a) one portion of fresh chicken breast 3 times a week, one of the following: one egg a day for patients below 40 kg, or two eggs a day for vegetarian patients and patients above 40 kg, 2 fresh peeled potatoes or 1 potato and 1 sweet potato daily, 1 banana, 1 apple (not cooked), and one additional fruit daily, selected from the group consisting of: 1 slice melon (cantaloupe or honeydew), fresh berries selected from the group consisting of: 5 strawberries, 5 raspberries or 10 blueberries, one portion of salad daily, comprising a combination of allowed vegetables, fruits and nuts;
(b) unlimited amounts of products selected from the group consisting of: white rice, rice noodles/rice papers, rice flour, lettuce/roquette, arugula, olive oil, canola oil, water, soda, herbal teas, chamomile tea, any home-made juice smoothie, salt, pepper, paprika, cinnamon stick, curcumin, turmeric, fresh herbs, fresh ginger, fresh lemon juice, fresh tomato sauce, unsweetened cocoa powder without additives, sulfite-free dark raisins for baking;
(c) no more by quantity than the following: Tofu 200 grams, once a week, 1 plain unflavored regular (3-4%) fat yoghurt daily, 1 additional egg, oatmeal, up to ½ a cup per day, wheat or spelt flour for baking, up to ½ cup once a week, 1 additional sweet potato daily, cooked quinoa, up to 4 tablespoons a day, legumes, selected from the group consisting of: lentils, peas, green beans, or dried chickpeas, up to 5 tablespoons a day, 1 slice of whole grain bread or ½ a cup of bread crumbs daily, cooked durum pasta, 200 grams, up to 2 times a week, one corn cob a day, fresh melon selected from the group consisting of cantaloupe, honeydew or watermelon, up to one slice daily, fresh berries selected from the group consisting of five strawberries, five raspberries or ten blueberries a day, one fresh fruit a day, selected from the group consisting of: 1 kiwi, 10 seedless grapes or 1 pear, 4 cubes of mango, 1 slice pineapple, 1 tangerine or one orange, 2 tomatoes daily, 2 cucumbers daily, 1 carrot daily, 1 zucchini daily, 3 broccoli or cauliflower florets or clusters daily, 1 avocado daily, 100 grams pumpkin, ½ red, yellow or green bell pepper a day, 4 fresh button mushrooms a day, 6 walnut halves or 6 almonds a day, one glass of freshly squeezed orange juice per day, pure honey for cooking or desserts, up to 3 tablespoons a day and table sugar for cooking or tea, up to 3 teaspoons a day;
thereby providing dietary components comprising food with reduced content of animal fat, dairy products, gluten, taurine, yeast, sulfites, and emsulsifiers.

64. The method of claim 63, wherein said limiting of food intake is carried out by a patient suffering from mild to moderate Ulcerative Colitis.

65. The method of claim 63, wherein said food products of said stage 1 and stage 2 are fresh.

66. The method of claim 63, wherein said food products may be processed in a manner selected from the group consisting of grilling, frying, baking, boiling, broiling, and any combination thereof.

67. The method of claim 63, wherein said stage 1 and said stage 2 additionally comprise the step of not consuming the following foods: red meat, pork, turkey, processed meat, seafood, fish, smoked meats and fish, all dairy products except unflavored yoghurt 3-4% fat, non-diary milk products, breads and baked goods of any kind, gluten free products not listed above, potato or corn flour, and any flour not listed as allowed, frozen potatoes or yams, corn during stage 1, legumes, comprising beans, chickpeas and soybeans during stage 1 and white beans during stage 2, margarine, any cooking oils except olive and canola oil, non-dairy creamers or cream, coconut oil, butter, dried fruits, canned fruits, all fruits not on the allowed list, all vegetables not on the allowed list, walnuts from mixes or trail mix, all nuts except unprocessed walnuts and almonds, soft drinks, fruit juices and sweetened beverages, alcoholic beverages, coffee, sauces, spreads, salad dressing, syrups and jams of any kind, canned products, packaged snacks, candy, chocolate, cakes, cookies, chewing gum, artificial sweeteners, mixed spices, ice-cream, yeast for baking.

68. A daily prepackaged therapeutic meal of a daily diet for consumption to be consumed daily for at least 12 weeks by an Ulcerative Colitis patient; said prepackaged therapeutic meal comprising food products for daily consumption; said food products comprising a first range of food products in said prepackaged therapeutic meal adapted to be consumed in weeks 0-6 and a second range of food products in said prepackaged therapeutic meal adapted to be consumed in weeks 7-12;
wherein said first range of food products comprise the following food products:
(a) at least one portion of fresh chicken breast three times a week, two peeled potatoes a day, 2 bananas a day, 1 apple a day, at least one portion of fresh salad a day from spinach or lettuce leaves with daily allowed fruit or vegetables, one of the following: one egg a day for patients below 40 kg, or two eggs a day for vegetarian patients and patients above 40 kg;
(b) unlimited amounts of products selected from the group consisting of: white rice, rice noodles/rice papers, rice flour, olive oil, canola oil, salt, pepper, paprika, cinnamon, curcumin, turmeric, fresh herbs, fresh ginger, fresh lemon juice, fresh tomato sauce, baking soda, water, soda, herbal teas, chamomile tea;
(c) no more by quantity than the following: tofu, 200 grams once a week from week 4, 3 additional eggs for cooking or baking, plain unflavored (3-4%) fat yoghurt 3 times a week, oatmeal up to ¼ cup (dry) a day, almond flour, up to ¼ cup weekly, cooked durum pasta, up to 200 grams, twice a week, cooked quinoa, up to 4 tablespoons a day, legumes, selected from the group consisting of:
lentils, peas or cooked green beans, up to 4 tablespoons a day, fresh melon selected from the group consisting of cantaloupe, honeydew or watermelon, up to one slice daily, fresh berries selected from the group consisting of five strawberries, five raspberries or ten blueberries a day, one fresh fruit selected from the group consisting of: 1 kiwi, 10 seedless grapes or 1 pear, 2 tomatoes daily, 2 cucumbers daily, 3 lettuce leaves daily, 1 cup spinach leaves daily, 1 carrot daily, 1 zuchinni daily, 3 broccoli or cauliflower florets or clusters daily, 1 avocado daily, 100 grams pumpkin daily, ½ red pepper daily, 6 unprocessed walnut halves or unprocessed unroasted almonds a day, one glass of freshly squeezed orange juice per day, pure honey for cooking or desserts, up to 3 tablespoons a day and table sugar for cooking or tea, up to 3 teaspoons a day;
further wherein said second range of food products comprise the following food products:
(a) one portion of fresh chicken breast 3 times a week, one of the following: one egg a day for patients below 40 kg, or two eggs a day for vegetarian patients and patients above 40 kg, 2 fresh potatoes or 1 potato and 1 sweet potato daily, 1 banana, 1 apple (not cooked), and one additional fruit daily, selected from the group consisting of: 1 slice melon (cantaloupe or honeydew), fresh berries (5 strawberries, 5 raspberries or 10 blueberries), one portion of salad daily, comprising a combination of allowed vegetables, fruits and nuts;
(b) unlimited amounts of products selected from the group consisting of: white rice, rice noodles/rice papers, rice flour, lettuce/roquette, arugula, olive oil, canola oil, water, soda, herbal teas, chamomile tea, any home-made juice smoothie, salt, pepper, paprika, cinnamon stick, curcumin, turmeric, fresh herbs, fresh ginger, fresh lemon juice, fresh tomato sauce, unsweetened cocoa powder without additives, sulfite-free dark raisins for baking;
(c) no more by quantity than the following: Tofu 200 grams, once a week, 1 plain unflavored regular (3-4%) fat yoghurt daily, 1 additional egg daily, oatmeal, up to ½ a cup per day, wheat or spelt flour for baking, up to ½ cup once a week, 1 additional sweet potato daily, cooked quinoa, up to 4 tablespoons a day, legumes, selected from the group consisting of: lentils, peas, green beans, or dried chickpeas, up to 5 tablespoons a day, 1 slice of whole grain bread, or ½ a cup of bread crumbs daily, cooked durum pasta, 200 grams, up to 2 times a week, one corn cob a day, fresh melon selected from the group consisting of cantaloupe, honeydew or watermelon, up to one slice daily, fresh berries selected from the group consisting of five strawberries, five raspberries or ten blueberries a day, one fresh fruit a day, selected from the group consisting of: 1 kiwi, 10 seedless grapes or 1 pear, 4 cubes of mango, 1 slice pineapple, 1 tangerine or one orange, 2 tomatoes daily, 2 cucumbers daily, 1 carrot daily, 1 zucchini daily, 3 broccoli or cauliflower florets or clusters daily, 1 avocado daily, 100 grams pumpkin, ½ red, yellow or green bell pepper a day, 4 fresh button mushrooms a day, 6 walnut halves or 6 almonds a day; one glass of freshly squeezed orange juice per day, pure honey for cooking or desserts, up to 3 tablespoons a day and table sugar for cooking or tea, up to 3 teaspoons a day;
thereby providing dietary components comprising food with reduced content of animal fat, dairy products, gluten, taurine, yeast, sulfites, and emulsifiers.

69. The daily prepackaged therapeutic meal of claim 68, wherein said daily prepackaged therapeutic meal is adapted to be provided to a patient suffering from mild to moderate Ulcerative Colitis.

70. The daily prepackaged therapeutic meal of claim 68, wherein said food products of said stage 1 and stage 2 are fresh.

71. The daily prepackaged therapeutic meal of claim 68, wherein said food products may be processed in a manner selected from the group consisting of grilling, frying, baking, boiling, broiling, and any combination thereof.

72. The daily prepackaged therapeutic meal of claim 68, wherein said stage 1 and said stage 2 additionally comprise the step of not consuming the following foods: red meat, pork, turkey, processed meat, seafood, fish, smoked meats and fish, all dairy products except unflavored yoghurt 3-4% fat, non-diary milk products, breads and baked goods of any kind, gluten free products not listed above, potato or corn flour, and any flour not listed as allowed, frozen potatoes or yams, corn during stage 1, legumes, comprising beans, chickpeas and soybeans during stage 1 and white beans during stage 2, margarine, any cooking oils except olive and canola oil, non-dairy creamers or cream, coconut oil, butter, dried fruits, canned fruits, all fruits not on the allowed list, all vegetables not on the allowed list, walnuts from mixes or trail mix, all nuts except unprocessed walnuts and almonds, soft drinks, fruit juices and sweetened beverages, alcoholic beverages, coffee, sauces, spreads, salad dressing, syrups and jams of any kind, canned products, packaged snacks, candy, chocolate, cakes, cookies, chewing gum, artificial sweeteners, mixed spices, ice-cream, yeast for baking.
